(19) United States
(12) Patent Application Publication
(10) Pub. No.: US 2024/0115247 A1
(43) Pub. Date: Apr. 11, 2024

(54) IMPROVED FORCEPS DEVICES

(71) Applicant: Michael J. Simoff, Northville, MI (US)

(72) Inventor: Michael J. Simoff, Northville, MI (US)

(21) Appl. No.: 18/546,453

(22) PCT Filed: Jan. 5, 2022
(86) PCT No.: PCT/US22/11274

§ 371 Date: Aug. 15, 2023

Related U.S. Application Data
(60) Provisional application No. 63/149,830, filed on Feb. 16, 2021.

(51) Int. Cl. A61B 10/06; A61B 10/04

(52) U.S. Cl. CPC A61B 10/06; A61B 10/04

(57) ABSTRACT

A forceps tool is usable with an endoscope or bronchoscope having an insertion tube defining a working channel. Components of the forceps tool include a flexible guide passable through, selectively extendable from, the working channel of the endoscope. A carrier extends outward from the flexible guide, such that the carrier extends further away from the endoscope. A first cup portion is configured to selectively catch and obtain tissue specimens and is pivotally attached to the carrier at a point distal to the flexible guide. The first cup portion is moveable or pivotable between a closed position and an open position. The open position is further away from the carrier than the closed position, as the first cup portion pivots about the distal point. The forceps tool may include a second cup portion. A method of using the forceps tool is also provided.

### CROSS-REFERENCE TO RELATED APPLICATIONS

This application claims the benefit of U.S. Provisional Application No. 63/149,830, filed Feb. 16, 2021, and PCT Application PCT/US22/11274, filed Jan. 5, 2022, both of which are hereby incorporated by reference in their entirety.

### INTRODUCTION

This disclosure generally relates to forceps tools and devices usable for collecting tissue samples, such as biopsies.

### SUMMARY

A forceps tool is provided. The forceps tool is usable with an endoscope or bronchoscope having an insertion tube defining a working channel. Components of the forceps tool include a flexible guide passable through the working channel of the endoscope. The flexible guide is selectively extendable from the working channel.

A carrier extends outward from the flexible guide, such that the carrier extends further away from the endoscope. A first cup portion is configured to selectively catch and obtain tissue specimens and is pivotally attached to the carrier at a point distal to the flexible guide.

The first cup portion is moveable or pivotable between a closed position and an open position. The open position is further away from the carrier than the closed position, as the first cup portion pivots about the distal point. The first cup has a first cup body and a first cam extending from the first cup body. A pivot may be formed on the carrier distal to the flexible guide, such that the first cam of the first cup portion is operatively attached to, and rotates about, the distal pivot.

The forceps tool may also include a second cup portion, which is configured to catch and obtain tissue specimens. The second cup portion is pivotally attached to the carrier and at a point distal to the flexible guide. The second cup portion includes a second cup body and a second cam extending from the cup body.

A method of collecting tissue samples with a forceps tool is also provided. The method includes moving a tip of an endoscope or bronchoscope to a location proximate to a tumor, and advancing the forceps tool, with a first cup portion closed, through the endoscope. The method further includes extending the forceps tool beyond the tip of the endoscope, and through the tumor, with the first cup portion closed, and then opening the first cup portion after it has passed through the tumor. The method also includes retracting the opened first cup portion into the tumor, and closing the first cup portion while at least partially within the tumor, such that a portion of the tumor is collected by the first cup portion. The forceps tool may then be retracted, with the collected portion of the tumor within the first cup portion, through the endoscope. The method may also be executed with a forceps tool having a second cup portion.

The above features and advantages, and other features and advantages, of the present disclosure are readily apparent from the following detailed description of some of the best modes and other embodiments for carrying out the disclosure, which is defined solely by the appended claims, when taken in connection with the accompanying drawings.

### BRIEF DESCRIPTION OF THE DRAWINGS

FIG. 1 schematically illustrates an isometric view of a forceps device or tool having a single cup, shown in an open position.

FIG. 2 schematically illustrates the single cup forceps device in a closed position and extending from a schematic endoscope.

FIG. 3 schematically illustrates an isometric view of a handle 18 usable with one or more forceps devices.

FIGS. 4A-4D schematically illustrate guide and carrier portions of the single cup forceps device. FIG. 4A shows an isometric or perspective view of the guide and carrier extending from the schematic endoscope; FIG. 4B shows a top view; FIG. 4C shows a back or end view with portions of the carrier element hidden; and FIG. 4D shows a side view.

FIGS. 5A-5D schematically illustrate a cup portion of the single cup forceps device. FIG. 5A shows an isometric view of the cup portion; FIG. 5B shows a top view; FIG. 5C shows a front or end view; and FIG. 5D shows a side view.

FIG. 6 schematically illustrates an isometric view of a forceps device or tool having a dual cup, shown in an open position.

FIG. 7 schematically illustrates the dual cup forceps device in a closed position.

FIGS. 8A-8D schematically illustrate guide and carrier portions of the dual cup forceps device. FIG. 8A shows an isometric or perspective view of the guide and carrier; FIG. 8B shows a top view; FIG. 8C shows a back or end view with portions of the carrier element hidden; and FIG. 8D shows a side view.

FIGS. 9A-9D schematically illustrate one cup portion of the dual cup forceps device. FIG. 9A shows an isometric view of the cup portion; FIG. 9B shows a top view; FIG. 9C shows a front or end view; and FIG. 9D shows a side view.

### DETAILED DESCRIPTION

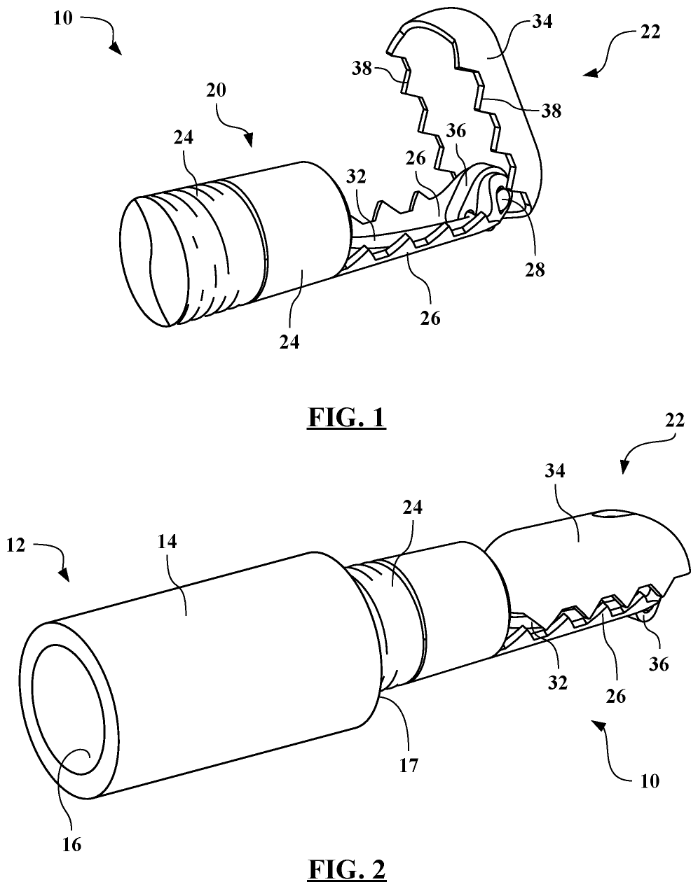

Referring to the drawings, like reference numbers correspond to like or similar components wherever possible throughout the several figures. All figures may be referred to in any section of the specification, without regard to numerical order. FIGS. 1 and 2 schematically illustrate a biopsy device, biopsy tool, or forceps tool 10 that is usable with an endoscope 12.

The forceps tool 10 shown in FIGS. 1 and 2 is a single cup device, as opposed to a multiple cup device. FIG. 1 shows the forceps tool 10 in an open position and FIG. 2 shows the forceps tool 10 in a closed position. The insertion tube 14 of the endoscope 12 defines an instrument channel or working channel 16, through which the forceps tool 10 may be inserted.

The portion of the endoscope 12 viewable in the figures is an insertion tube 14, which is shown highly schematically and extends much further leftward (as viewed in FIG. 2) relative to the forceps tool 10. The endoscope 12 is of sufficient design and length to pass through the nose or mouth and is configured to access the airways of the lungs. The working channel 16 passes through the length of the endoscope 12. It allows the passage of tools safely through the length of the endoscope when in place within the lungs.

Those having ordinary skill in the art will recognize numerous types and configurations for the endoscope 12, which may be referred to as a bronchoscope, usable with the forceps tool 10 described herein. The working channel 16 ends at a tip 17, beyond which the forceps tool 10 is selectively extendable. Note that the forceps tool 10 would extend leftward (as viewed in FIG. 2) through the working channel 16 of the insertion tube 14, but that portion is not illustrated in order to better illustrate the working channel 16 through which the forceps tool 10 passes.

Referring also to FIG. 3, the biopsy device or forceps tool 10 may be triggered or controlled by a handle 18, which is shown schematically in FIG. 3. Skilled artisans will recognize use of the handle 18 with the endoscope 12 to selectively operate the forceps tool 10 described herein. The endoscope 12 may be robotic, manually operated, or otherwise controlled.

For example, and without limitation, there are generally 4 ways of accessing a nodule to biopsy it. The first is a robotic system and the second is a manual system, where, using a bronchoscope, the device is inserted through, and into, the periphery of the lung, typically using fluoroscopy. The third is called electromagnetic navigation (EMN), and the fourth is called radial endobronchial ultrasound (rEBUS) guided procedure.

With EMN, a navigational catheter is inserted through a bronchoscope or endoscope, with which the user can partially control the direction of the tip towards the lesion to be sampled. Once guided to the location of the lesion, the navigational catheter is removed and an extendable biopsy channel (a hollow catheter) is left in place, through which the forceps tool 10 and/or other tools are placed. With rEBUS, a radial ultrasound probe with a catheter is passed through a manual bronchoscope and pushed out into the lung. When moved to the correct location, the ultrasound probe is removed, and the catheter left in place.

The forceps tool 10, as delivered and aligned by the endoscope 12, may be used to collect tissue samples, such as those used to test for one or more diseases. For example, and without limitation, the forceps tool 10 may be used to perform a biopsy of a tumor, lesion, or nodule to evaluate for lung cancer.

Bronchoscopy—the use of a fiberoptic scope inserted through the mouth or nose of a patient—is one technique to access lung nodules for the diagnosis of lung cancer. The traditional procedure of bronchoscopy used to sample a peripheral lesion is a transbronchial biopsy, which uses forceps directed out into the lung to biopsy a lesion. The endoscope 12 may be a robotic bronchoscope, which provides a stable and sensitive platform for the forceps tool 10. The robotic endoscope 12 can be used to guide the forceps tool 10 to and localize peripheral tumors to a higher percentage of capture.

A variety of imaging technologies are used in the assessment of patients with suspected lung cancer, often identifying sites of disease or assisting in choosing targets for biopsy. These technologies may be used in conjunction with the forceps tool 10 described herein.

While the present disclosure may be illustrated with respect to particular industries or applications, those skilled in the art will recognize the broader applicability of the products, methods, and techniques, described herein. For example, similar structures, methods, or combinations thereof, may be used in other industries or for medical procedures other than those described herein.

Those having ordinary skill in the art will also recognize that terms such as “above,” “below,” “upward,” “downward,” et cetera, are used descriptively of the figures, and do not represent limitations on the scope of the appended claims. Any numerical designations, such as “first” or “second” are illustrative only and are not intended to limit the scope of the claims in any way.

When used herein, the term “substantially” refers to relationships that are ideally perfect or complete, but where manufacturing realties prevent absolute perfection. Therefore, substantially denotes typical variance from perfection in the relevant art. For example, if height A is substantially equal to height B, it may be preferred that the two heights are 100.0% equivalent, but manufacturing realities likely result in the distances varying from such perfection. Skilled artisans would recognize the amount of acceptable variance. For example, and without limitation, coverages, areas, or distances may generally be within 10% of perfection for substantial equivalence. Similarly, relative alignments, such as parallel or perpendicular, may generally be within 5%.

Features shown in one figure may be combined with, substituted for, or modified by, features shown in any of the figures. Unless stated otherwise, no features, elements, or limitations are mutually exclusive of any other features, elements, or limitations. Any specific configurations shown in the figures are illustrative only and the specific configurations shown are not limiting. Any use of the term, “or,” whether in the description or claims, is inclusive of any specific element referenced and, also, includes any combination of the elements referenced, unless otherwise explicitly stated.

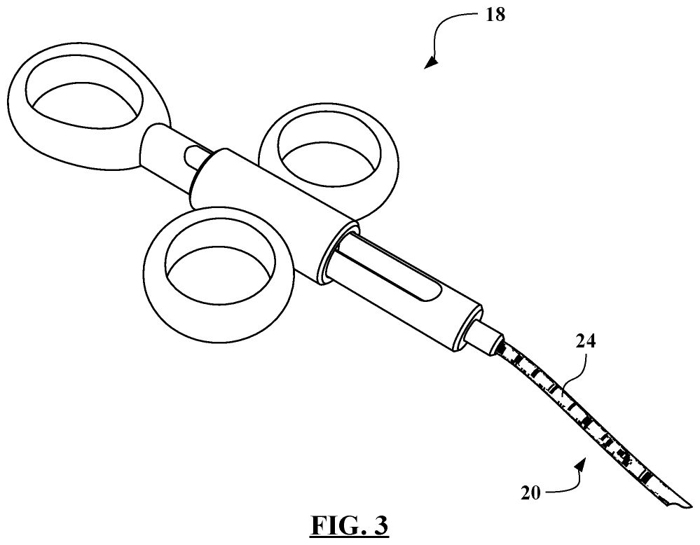

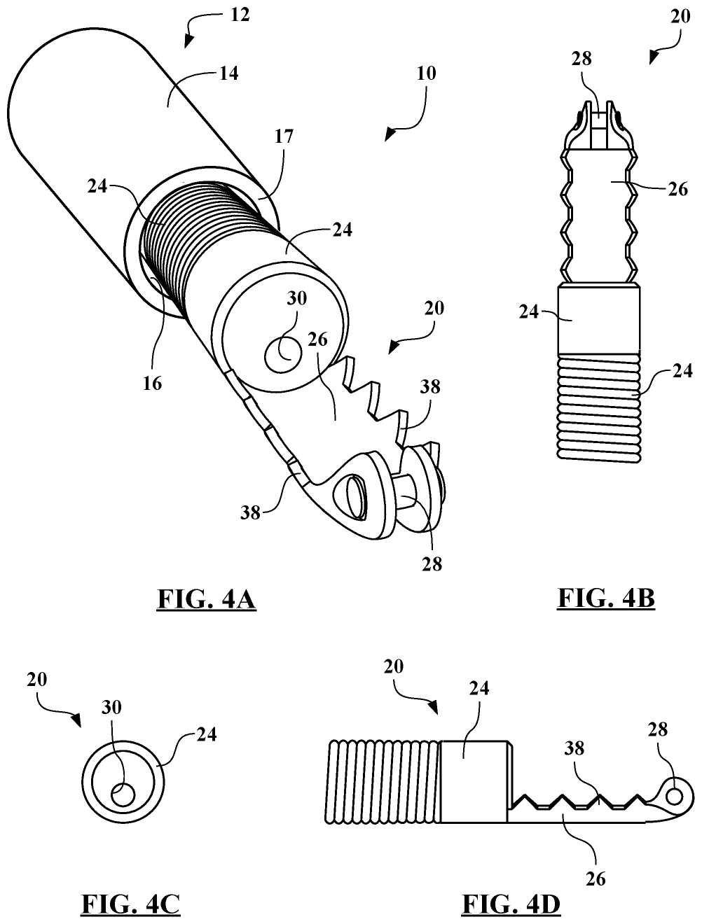

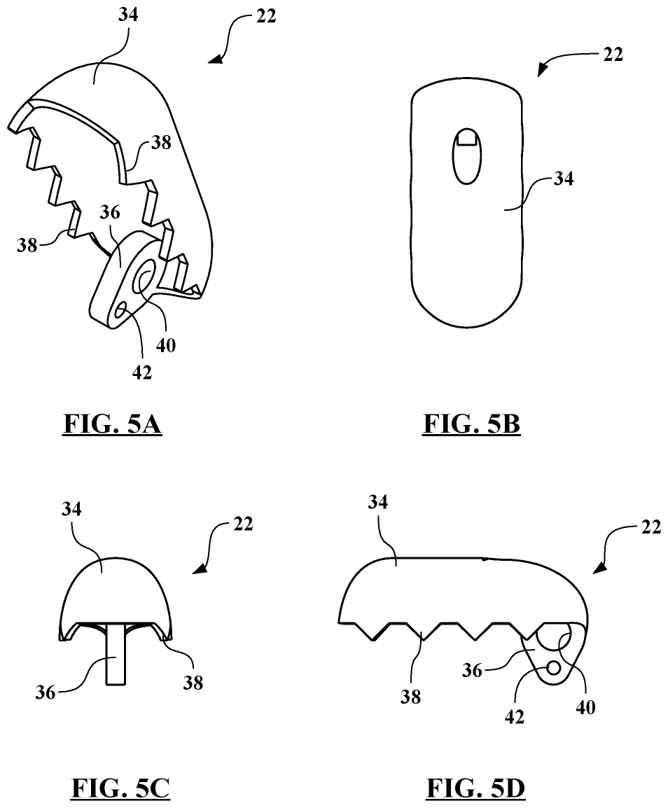

The forceps tool 10 includes a guide portion or flexible guide 20 and a cup portion 22, as shown in FIGS. 1 and 2. FIGS. 4A-4D show portions of the flexible guide 20 in more detail and from different viewpoints, and FIGS. 5A-5D show portions of the cup portion 22 in more detail and from different viewpoints.

The flexible guide 20 includes a flexible tube 24 that is passable through the endoscope 12. The flexible guide 20 is selectively extendable from, and moveable within, the working channel 16 of the endoscope 12. Note that, in most configurations, the forceps tool 10 will only be passable through the working channel 16 of the endoscope 12 when the cup portion 22 is closed, as shown in FIG. 2.

A carrier 26 extends outward from the flexible tube 24, such that the carrier 26 extends further away from the working channel 16 of the endoscope 12. A pivot or pivot bar 28 is defined, formed, or held by the carrier 26. Note that the pivot bar 28 is distal to the flexible tube 24. The pivot bar 28 cooperates with the cup portion 22 to open and close the cup portion 22.

A cable channel 30 is formed through a portion of the flexible guide 20, and possibly a portion of the carrier 26, depending on the configuration. A pull cable 32 passes through the cable channel 30 in the flexible tube 24 and is configured to selectively move or actuate the cup portion 22. The pull cable 32 is operatively attached to the handle 18, such that portions of the handle 18 are configured to trigger or control movement of the cup portion 22 via the pull cable 32.

The cup portion 22 is configured to catch and obtain tissue specimens and is pivotally attached to the carrier 26 at the far end of the flexible guide 20. The cup portion 22 is moveable between a closed position (shown in FIG. 2) and an open position (shown in FIG. 1). Note that the open position is further away from the flexible tube 24 than the closed position, such that the cup portion 22 is closer to the distal end of the forceps tool 10 in the open position.

The cup portion 22 has a cup body 34 and a cam 36 extending from the cup body 34. In the configuration shown, one or more teeth 38 extend from the cup body 34. However, other configurations may not include the teeth 38, such that the edge of the cup body 34 is generally smooth or has a chamfered edge for cutting. For the single cup configuration of the forceps tool 10 shown in the figures, the carrier 26 also defines a cup-like portion that has additional teeth 38.

A pivot point 40 is defined in the cam 36, and a pull attachment 42 is defined in the cam 36, opposite from the cup body 34. The pivot bar 28 and the pivot point 40 form a pivot mechanism that is configured to allow the cup portion 22 to move between the closed position and the open position in response to the pull cable 32 moving the pull attachment 42.

The handle 18 and the pull cable 32, possibly in cooperation with other components and elements, operate as a control mechanism causing the cup portion 22 to pivot between the closed position and the open position, and back to the closed position. The cup body 34 is curved toward the carrier 26 and rotates open about the pivot point 40 toward the distal end of the carrier 26, such that the cup portion 22 forms an inward-opening area for collecting tissue samples.

The forceps tool 10 shown in the figures may be described as a reverse-opening forceps. The forceps tool 10 may be used to collect tissue samples, such as for biopsy purposes. Alternative forceps devices, forward-opening forceps, open one or more cups with the opened portion facing away from the endoscope of the alternative biopsy device—i.e., the cups rotate opposite the direction illustrated in FIGS. 1 and 2.

Therefore, the alternative forceps push away from the scope and directly towards lung structures. During many usages of forward-opening forceps, as opposed to the reverse-opening forceps tool 10, the lesion or tissue targeted for sampling is pushed away from the forward-opening forceps. When forward-opening forceps are placed through a scope, they must be extended 7-10 mm beyond the tip before they can be opened, such that they are pushed outward from the scope. After extending and opening the forward-opening forceps, the scope tip must be repositioned, which may introduce error in the position of the scope tip and/or the forward-opening forceps relative to the lesion. In the soft tissue environment of the lung, as the forward-opening forceps extends away from the scope tip, the tumor may be pushed backward, or to the side, decreasing the effectiveness of biopsy.

Ineffective biopsy will result in inadequate tissue acquisition requiring repeated biopsy procedures at a later date, delaying the diagnosis of cancer and often affecting treatment outcome. Furthermore, by pushing the lesion away from the forward-opening forceps, into the periphery of the lung, there is an increased risk of grasping tissue that is beyond the tumor, including, but not limited to, the pleura or mediastinal structures, such that those tissues or structures may be damaged.

Contrarily, the reverse-opening forceps tool 10 offers a unique approach for biopsy of lesions or tumors. In some configurations, due to the robotic platform of the endoscope 12, the lesions may be aligned closer to the tip 17 of the endoscope 12 than with previously used technologies, which required space for opening. For example, and without limitation, the tip 17 may be located less than 5 mm from the lesion to be sampled. In many cases, the tip 17 may actually abut the lesion. When in position, the robotic endoscope 12 also provides a semi-rigid platform keeping the tip 17 of the endoscope 12 in position relative to the tumor. This allows the tumor to be positioned between the cup portion 22 and the tip 17 of the endoscope 12, allowing more accurate and efficient tissue biopsy of lesions.

In one example of operation of the forceps tool 10 to biopsy a tumor, the forceps tool 10 is advanced, away from the endoscope 12, through the tumor with the cup portion 22 closed. The cup portion 22 may then be opened after exiting the other side of the tumor. Alternatively, the forceps tool 10 may be opened after partially entering the tumor. The cup portion 22 may then be retracted through the tumor, with the tumor being stabilized by the tip 17 of the endoscope 12. The cup portion 22 may be closed while the tumor is at least partially pressed against the tip 17 of the endoscope 12 for stabilization. The cup portion 22 is closed with an inwardly sweeping motion that captures portions of the lesion without pushing it away from the remainder of the forceps tool 10 and the endoscope 12.

After closing the cup portion 22, with the sample is captured within the cup body 34, the forceps tool 10 may be retracted into the working channel 16 of the endoscope 12 with the biopsied sample contained therein. This process, enabled by the structures of the forceps tool 10 shown in the figures, allows improved sampling and makes the likelihood of an accurate diagnosis more likely.

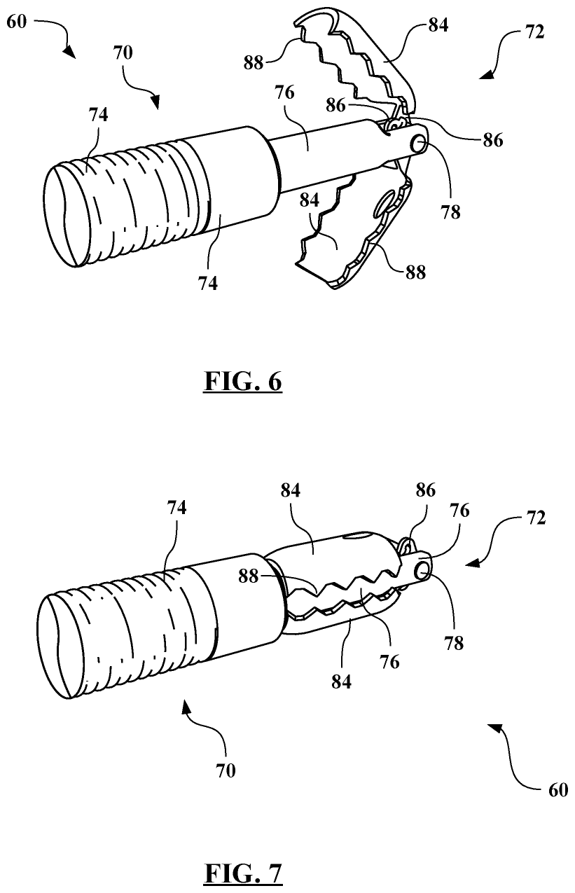

FIGS. 6 and 7 show a dual cup version of a biopsy device or forceps tool 60. FIG. 6 shows the dual cup forceps tool 60 in an open position and FIG. 7 shows the dual cup forceps tool 60 in a closed position.

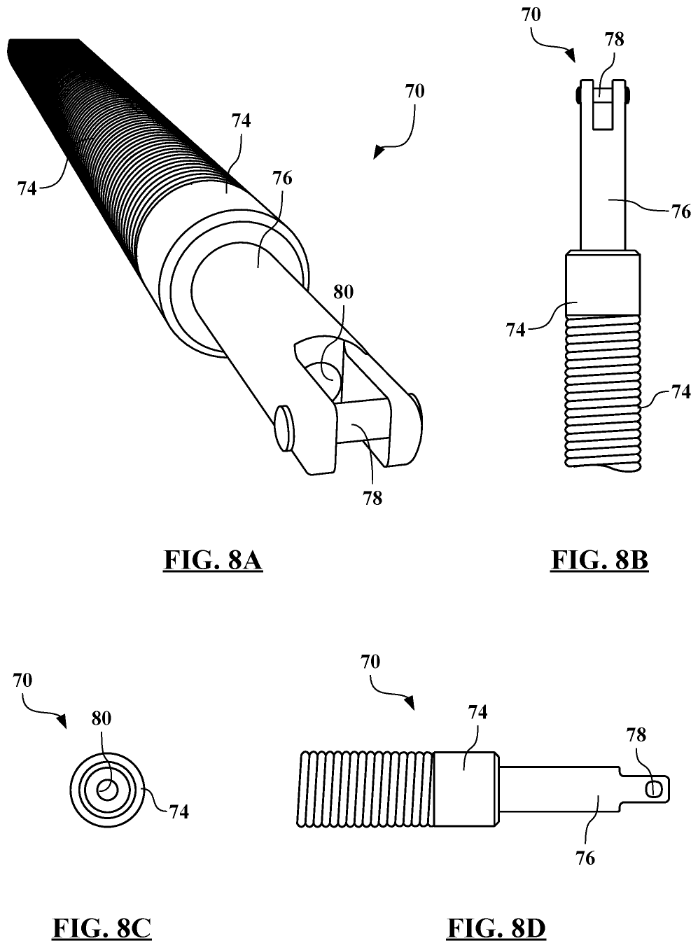

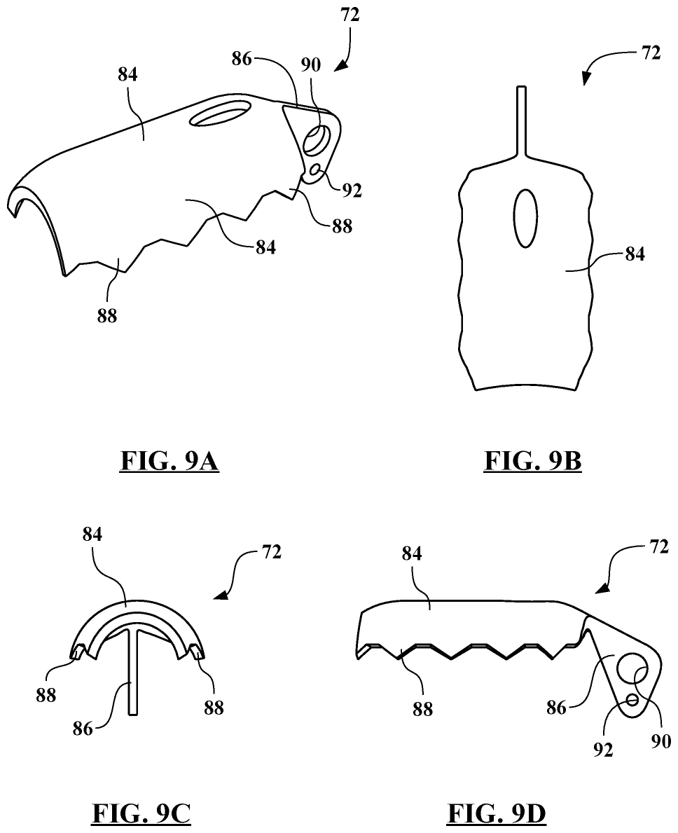

The forceps tool 60 includes a guide portion or flexible guide 70 and two cup portions 72, which may be referred to as first and second cup portions, as shown in FIGS. 6 and 7. FIGS. 8A-8D show portions of the flexible guide 70 in more detail and from different viewpoints, and FIGS. 9A-9D show one of the cup portions 72 in more detail and from different viewpoints.

The flexible guide 70 has a flexible tube 74 that is passable through an endoscope, such as the endoscope 12 or another suitable device. The flexible guide 70 is selectively extendable from, and moveable within, the working channel 16 of the endoscope 12 when the cup portions 72 are closed. A carrier 76 extends outward from the flexible tube 74, such that the carrier 76 extends further away from the working channel 16 of the endoscope 12.

A pivot or pivot bar 78 is defined, formed, or held by the carrier 76. Note that the pivot bar 78 is distal to the flexible tube 74. The pivot bar 78 cooperates with the cup portions 72 to open and close the cup portions 72.

A cable channel 80 is formed through the flexible guide 70, including portions of the carrier 76. A pull cable, which is largely hidden from view in the figures, passes through the cable channel 80 in the flexible tube 74 and is configured to selectively move or actuate the cup portions 72. The pull cable is operatively attached to a handle, such as the handle 18 or other suitable structure, such that the handle 18 may be configured to trigger movement of the cup portions 72.

The cup portions 72 are configured to catch and obtain tissue specimens and are pivotally attached to the carrier 76, such as via the pivot bar 78, at the far end of the flexible guide 70. The cup portions 72 are moveable between a closed position (shown in FIG. 7) and an open position (shown in FIG. 6). Note that the open position is further away from the flexible tube 74 than the closed position, such that the cup portions 72 are nearer the distal end of the forceps tool 60 in the open position.

The two cup portions 72 illustrated in the figures may be very similar to each other and have a cup body 84 and a cam 86 extending from the cup body 84. The pull cable, which may be split into two cables, is operatively attached to the cams 86. However, note that differing cup shapes may be used for the two cup portions 72, and the carrier 76 may be shaped with recessed portions to indentations to further capture tissue samples.

In the configuration shown, one or more teeth 88 extend from the cup body 84. However, other configurations may not include the teeth 88, such that the edge of the cup body 84 is generally smooth or has a chamfered edge to assist with cutting into the lesion. The cams 86 of the cup portions 72 may be slightly offset from the center of the cup body 84, such that the two cams 86 are configured to rotate next to one another. Alternatively, the cams 86 may be separated or spaced apart by a bearing or bushing, which may assist actuation via the pull cable.

A pivot point 90 is defined in each of the cams 86, and a pull attachment 92 is defined in each of the cams 86, opposite the cup bodies 84. The pivot bar 78 and the pivot points 90 form pivot mechanisms that are configured to allow the cup portions 72 to move between the closed position and the open position in response to the pull cable moving the pull attachments 92 and the cams 86. Note that the pull cable of the dual cup forceps tool 60 may split or branch at its end, in order to operatively connect to both of the pull attachments 92.

The handle 18 and the pull cable, possibly in cooperation with other components, operate as a control mechanism causing the cup portions 72 to pivot between the closed position and the open position. The cup bodies 84 are curved toward the carrier 76 and rotate open about the pivot point 90 toward the distal end of the carrier 76, such that the cup portions 72 form inward-opening areas for collecting tissue samples.

Closing the cup portions 72 at least partially within the lesion collects samples inward toward the carrier 76, the flexible tube 74, and the endoscope 12. The closed forceps tool 60 may then be retracted through the endoscope 12 with the biopsied sample contained therein. The endoscope 12 provides a stable and rigid base for the forceps tool 60 to sample the associated tumor/lesion/nodule.

In some configurations, the forceps tool 10 may be used with devices other than the endoscope 12 or a robotic bronchoscope and/or may be used for sampling lesions not involved with the lungs. For example, and without limitation, the forceps tool 10 may be used for gastrointestinal procedures (GI) to sample lesions within the GI tract.

Clauses follow.
1. A forceps tool usable with an endoscope having an insertion tube defining a working channel, the forceps tool comprising: a flexible guide passable through the working channel of the endoscope, and selectively extendable therefrom; a carrier extending outward from the flexible guide, such that the carrier extends away from the endoscope; and a first cup portion, configured to catch and obtain tissue specimens, pivotally attached to the carrier at a point distal to the flexible guide, wherein the first cup portion is moveable between a closed position and an open position, and wherein the first cup portion is further away from the carrier in the open position than the closed position.
2. The forceps tool of claim 1, wherein the first cup portion further includes: a first cup body; and a first cam extending from the first cup body.
3. The forceps tool of claim 2, further comprising: a pivot formed on the carrier distal to the flexible guide, wherein the first cam of the first cup portion is operatively attached to, and rotates about, the pivot.
4. The forceps tool of claim 3, further comprising: a control mechanism operatively attached to the first cup portion and causing the first cup portion to rotate between the closed position and the open position.
5. The forceps tool of claim 4, wherein the control mechanism includes a cable actuated by a handle.
6. The forceps tool of claim 5, further comprising: a second cup portion, configured to catch and obtain tissue specimens, pivotally attached to the carrier and at a point distal to the flexible guide, and having: a second cup body; and a second cam extending from the second cup body.
7. The forceps tool of claim 6, further comprising: wherein the first cam of the first cup portion is operatively attached to, and rotates about, the pivot, and wherein the second cam of the second cup portion is operatively attached to, and rotates about, the pivot.
8. The forceps tool of claim 2, further comprising: a second cup portion, configured to catch and obtain tissue specimens, pivotally attached to the carrier and at a point distal to the flexible guide, and having: a second cup body; and a second cam extending from the second cup body.
9. The forceps tool of claim 8, further comprising: a pivot formed on the carrier distal to the flexible guide, wherein the first cam of the first cup portion is operatively attached to, and rotates about, the pivot, and wherein the second cam of the second cup portion is operatively attached to, and rotates about, the pivot.
10. A forceps tool comprising: a flexible guide; a carrier extending outward from the flexible guide; and a first cup portion, configured to catch and obtain tissue specimens, pivotally attached to the carrier at a point distal from the flexible guide, wherein the first cup portion is moveable between a closed position and an open position, wherein the first cup portion is further away from the carrier in the open position than the closed position.
11. The forceps tool of claim 10, further comprising: a first cup body formed on the first cup portion; a first cam extending from the first cup body; and a pivot formed on the carrier distal to the flexible guide, wherein the first cam of the first cup portion is operatively attached to, and rotates about, the pivot of the carrier.
12. The forceps tool of claim 11, further comprising: a second cup portion, configured to catch and obtain tissue specimens, pivotally attached to the carrier at the pivot, and having: a second cup body; and a second cam extending from the second cup body, wherein the second cam of the second cup portion is operatively attached to, and rotates about, the pivot, such that wherein the second cup portion is moveable between a closed position and an open position and the second cup portion is further away from the carrier in the open position than the closed position.
13. The forceps tool of claim 12, further comprising: a control mechanism operatively attached to the first cup portion and the second cup portion, and configured to cause the first cup portion and the second cup portion to rotate between the respective closed position and the open position.
14. A method of collecting tissue samples with an endoscope and a forceps tool, the method comprising: moving a tip of the endoscope to a location proximate to a tumor; advancing the forceps tool, with a first cup portion closed, through the endoscope; extending the forceps tool beyond the tip of the endoscope, and through the tumor, with the first cup portion closed; opening the first cup portion after the first cup portion of the forceps tool has passed through the tumor, wherein opening the first cup portion causes the first cup portion to move further away from the tip of the endoscope; retracting the opened first cup portion into the tumor; closing the first cup portion while at least partially within the tumor, such that a portion of the tumor is collected by the first cup portion; and retracting the forceps tool, with the collected portion of the tumor within the first cup portion, through the endoscope.
15. The method claim 14, wherein the tip of the endoscope is placed within five millimeters of the tumor.
16. The method claim 15, wherein the forceps tool includes a second cup portion, the method comprising: advancing the forceps tool, with the first cup portion and the second cup portion closed, through the endoscope; extending the forceps tool beyond the tip of the endoscope, and through the tumor, with the first cup portion and the second cup portion closed; opening the first cup portion and the second cup portion after the first cup portion and the second cup portion have passed through the tumor, wherein opening the first cup portion and the second cup portion causes both the first cup portion and the second cup portion to move further away from the tip of the endoscope; retracting the opened first cup portion and the second cup portion into the tumor; closing the first cup portion and the second cup portion while at least partially within the tumor, such that a portion of the tumor is collected by the first cup portion and the second cup portion; and retracting the forceps tool, with the collected portion of the tumor within the first cup portion and the second cup portion, through the endoscope.

The detailed description and the drawings or figures are supportive and descriptive of the disclosure. While some of the best modes and other embodiments for carrying out the disclosure have been described in detail, various alternative designs, configurations, and embodiments exist for practicing the appended claims, as will be recognized by those having ordinary skill in the art.

1. A forceps tool usable with an endoscope having an insertion tube defining a working channel, the forceps tool comprising:
a flexible guide passable through the working channel of the endoscope, and selectively extendable therefrom;
a carrier extending outward from the flexible guide, such that the carrier extends away from the endoscope; and
a first cup portion, configured to catch and obtain tissue specimens, pivotally attached to the carrier at a point distal to the flexible guide,
wherein the first cup portion is moveable between a closed position and an open position, and wherein the first cup portion is further away from the carrier in the open position than the closed position.

2. The forceps tool of claim 1, wherein the first cup portion further includes:
a first cup body; and
a first cam extending from the first cup body.

3. The forceps tool of claim 2, further comprising:
a pivot formed on the carrier distal to the flexible guide,
wherein the first cam of the first cup portion is operatively attached to, and rotates about, the pivot.

4. The forceps tool of claim 3, further comprising:
a control mechanism operatively attached to the first cup portion and causing the first cup portion to rotate between the closed position and the open position.

5. The forceps tool of claim 4, wherein the control mechanism includes a cable actuated by a handle.

6. The forceps tool of claim 5, further comprising:
a second cup portion, configured to catch and obtain tissue specimens, pivotally attached to the carrier and at a point distal to the flexible guide, and having:
a second cup body; and
a second cam extending from the second cup body.

7. The forceps tool of claim 6, further comprising:
wherein the first cam of the first cup portion is operatively attached to, and rotates about, the pivot, and
wherein the second cam of the second cup portion is operatively attached to, and rotates about, the pivot.

8. The forceps tool of claim 2, further comprising:
a second cup portion, configured to catch and obtain tissue specimens, pivotally attached to the carrier and at a point distal to the flexible guide, and having:
a second cup body; and
a second cam extending from the second cup body.

9. The forceps tool of claim 8, further comprising:
a pivot formed on the carrier distal to the flexible guide,
wherein the first cam of the first cup portion is operatively attached to, and rotates about, the pivot, and
wherein the second cam of the second cup portion is operatively attached to, and rotates about, the pivot.

10. A forceps tool comprising:
a flexible guide;
a carrier extending outward from the flexible guide; and
a first cup portion, configured to catch and obtain tissue specimens, pivotally attached to the carrier at a point distal from the flexible guide,
wherein the first cup portion is moveable between a closed position and an open position, wherein the first cup portion is further away from the carrier in the open position than the closed position.

11. The forceps tool of claim 10, further comprising:
a first cup body formed on the first cup portion;
a first cam extending from the first cup body; and
a pivot formed on the carrier distal to the flexible guide,
wherein the first cam of the first cup portion is operatively attached to, and rotates about, the pivot of the carrier.

12. The forceps tool of claim 11, further comprising:
a second cup portion, configured to catch and obtain tissue specimens, pivotally attached to the carrier at the pivot, and having:
a second cup body; and
a second cam extending from the second cup body,
wherein the second cam of the second cup portion is operatively attached to, and rotates about, the pivot, such that wherein the second cup portion is moveable between a closed position and an open position and the second cup portion is further away from the carrier in the open position than the closed position.

13. The forceps tool of claim 12, further comprising:
a control mechanism operatively attached to the first cup portion and the second cup portion, and configured to cause the first cup portion and the second cup portion to rotate between the respective closed position and the open position.

14. A method of collecting tissue samples with an endoscope and a forceps tool, the method comprising:
moving a tip of the endoscope to a location proximate to a tumor;
advancing the forceps tool, with a first cup portion closed, through the endoscope;
extending the forceps tool beyond the tip of the endoscope, and through the tumor, with the first cup portion closed;
opening the first cup portion after the first cup portion of the forceps tool has passed through the tumor, wherein opening the first cup portion causes the first cup portion to move further away from the tip of the endoscope;
retracting the opened first cup portion into the tumor;
closing the first cup portion while at least partially within the tumor, such that a portion of the tumor is collected by the first cup portion; and
retracting the forceps tool, with the collected portion of the tumor within the first cup portion, through the endoscope.

15. The method claim 14, wherein the tip of the endoscope is placed within five millimeters of the tumor.

16. The method claim 15, wherein the forceps tool includes a second cup portion, the method comprising:
advancing the forceps tool, with the first cup portion and the second cup portion closed, through the endoscope;
extending the forceps tool beyond the tip of the endoscope, and through the tumor, with the first cup portion and the second cup portion closed;
opening the first cup portion and the second cup portion after the first cup portion and the second cup portion have passed through the tumor, wherein opening the first cup portion and the second cup portion causes both the first cup portion and the second cup portion to move further away from the tip of the endoscope;
retracting the opened first cup portion and the second cup portion into the tumor;
closing the first cup portion and the second cup portion while at least partially within the tumor, such that a portion of the tumor is collected by the first cup portion and the second cup portion; and
retracting the forceps tool, with the collected portion of the tumor within the first cup portion and the second cup portion, through the endoscope.
